(19) United States
(12) Patent Application Publication
(10) Pub. No.: US 2026/0182977 A1
(43) Pub. Date: Jul. 2, 2026

(54) DEVICE FOR DELIVERY OF SURGICAL PATCHES DURING MINIMALLY INVASIVE SURGERY

(71) Applicant: SEALANTIUM MEDICAL LTD, Rosh Ha'Ayin (IL)

(72) Inventors: Orgad LAUB, Tel Aviv (IL); Eran COHEN, Hod Hasharon (IL); Yotam SCHWARTZ, Mishmar Ayalon (IL); Yonatan NAVEH, Hod Hasharon (IL); Hagay WEISBROD, Jordan Valley (IL); Yaron FUERST, Kfar Vradim (IL)

(21) Appl. No.: 19/129,301

(22) PCT Filed: Nov. 14, 2023
(86) PCT No.: PCT/IL2023/051177

§ 371 Date: May 13, 2025

Related U.S. Application Data
(60) Provisional application No. 63/424,982, filed on Nov. 14, 2022.

(51) Int. Cl. A61B 17/00; A61B 50/00; A61B 50/33

(52) U.S. Cl. CPC A61B 17/0057; A61B 17/00234; A61B 50/33; A61B 2017/00575; A61B 2017/00597; A61B 2017/00623; A61B 2017/0065; A61B 2017/00659; A61B 2017/00862; A61B 2017/00951; A61B 2050/0065

(57) ABSTRACT

A device for introduction and delivery of a self-adhesive surgical patch during minimally invasive surgery is disclosed. The device comprises a base that can be compressed to fit inside a 5 mm i.d. tubular member. In preferred embodiments of the invention, the base comprises a frame made of a compliant material. The patch is attached to the frame while the frame is in a planar open configuration. Pressure is then applied to opposite sides of the frame, compressing it to a linear closed configuration small enough to pass through a 5 mm i.d. tubular member. After passing through the tubular member, the frame is reopened to its open configuration, enabling delivery and attachment of the patch to a target tissue. A kit for providing the device is also disclosed.

### CROSS-REFERENCE TO RELATED PUBLICATIONS

This application claims priority from U.S. Provisional Pat. Appl. No. 63/424,982, filed 14 Nov. 2022.

### FIELD OF THE INVENTION

This invention relates in general to devices for use in minimally invasive surgery. It relates more specifically to devices for delivering self-sealing surgical patches such as hemostatic patches during minimally invasive surgery and methods for implementing their use.

### BACKGROUND OF THE INVENTION

Self-sealing surgical patches such as hemostatic patches are used during minimally invasive surgery such as laparoscopic surgery to seal wounds, and to create hemostasis by sealing a tissue or organ. Numerous devices and methods have been developed for delivery of self-sealing patches to a desired site.

U.S. Pat. No. 5,397,332 discloses an applicator for a sheet of surgical material such as a surgical patch. The applicator comprises an expandable spreader tip that is normally housed in a collapsed configuration within delivery tube. The elongated tube is introduced into the body. The delivery tube is retracted, and an actuator expands the spreader tip, which is then used to apply the surgical material. The spreader tip is made of stainless steel.

U.S. Pat. No. 10,595,978 discloses a method of delivering a hemostatic patch to internal tissue during minimally invasive surgery. The patch is rolled up and placed within a plastic bag. The plastic bag containing the patch is then inserted into a body cavity via an elongated tube. The plastic bag is removed and the patch then unrolled and applied to the desired tissue.

U. S. Pat. Appl. Pub. No. 2003/0073979 discloses a device for delivering patches. The device comprises an umbrella- or basket-like expandable assembly. The assembly is kept in its retracted form inside a delivery tube. When the device is extended from the delivery tube, it expands into its normal form, in which it is used to deliver a patch attached to it to tissue. In some embodiments of the device, it is self-expanding. The expanding elements can be made from wire constructed from a material such as nitinol, stainless steel, platinum, or other materials with super-elastic characteristics.

U.S. Pat. Appl. Pub. No. 2009/0030435 discloses a device for anchoring cardiovascular implants. In some embodiments of the invention, the anchor is expandable upon deployment; it is held in a cannula or tube in a compressed form and then expands after being expelled from the tube. The anchor can be made of “standard” materials such as stainless steel or nitinol.

U.S. Pat. Appl. Pub. No. 2014/0277579 discloses a construct for use in minimally invasive surgery. The construct comprises an allograft patch having at least one layer of human amnion and chorion tissues and a collapsible rigid or semi-rigid frame. The patch is attached to the frame, which expands into its final shape after exiting the cannula through which it is inserted into the patient's body, enabling the patch to be applied to the tissue. Preferred shapes for the frame include a semi-spherical shape, a plurality of spokes, and a cylinder.

International (PCT) Pat. Appl. Pub. No. WO2011128903 discloses a device for delivery and positioning of a spreadable sheet in minimally invasive surgery. The device includes a self-expanding assembly that may comprise a plurality of spokes that in the collapsed configuration bend back over a guide rod. The spreadable sheet is attached to the expandable end, and the device is inserted into a tube. When the device exits the tube, the assembly expands, enabling delivery and positioning of the spreadable sheet.

International (PCT) Pat. Appl. Pub. No. WO2015111040 discloses a device for use in ocular surgery, in particular, for removal of Descemet's membrane. The device includes a semi-rigid circular frame that may be made of any material that will allow it to hold its shape; plastics and metal alloys are given as example. The frame passes through a conical inserter head in a folded form; once the frame exits the inserter, it expands into its final circular shape; typical dimensions of 0.5 mm thickness and 9-10 mm diameter are given.

From the foregoing, it is clear that while a number of devices and techniques have been developed for delivery of surgical patches during minimally invasive surgery, the devices and methods known in the art tend to be complicated and awkward for the surgeon. Development of improved devices and methods for delivery of self-adhesive tissue patches, in particular hemostatic patches, in minimally-invasive surgery thus remains a long-felt, but as yet unmet need.

### SUMMARY OF THE INVENTION

The invention disclosed herein is designed to meet this need. Devices and methods for introduction of a self-adhesive tissue patch are disclosed in which the device and patch can be introduced via a tubular member typically used in minimally-invasive surgery such as a trocar; the device can be withdrawn through the same tubular member after application of the patch; the patch is not activated prematurely; and the entire process of introduction of the device and patch, attachment of the patch to the desired tissue and organ, and withdrawal of the device, can be performed using only standard graspers use in minimally-invasive surgery.

It is therefore one object of this invention to disclose a device for introducing a self-adhesive surgical patch during minimally invasive surgery, said device comprising a flexible base, wherein said base is sufficiently flexible and comprises members of dimensions sufficiently small such that said base can be compressed to a size sufficiently small to be capable of insertion into a tubular member designed for use in minimally-invasive surgery.

It is a further object of this invention to disclose such a device, wherein said base comprises a frame (100) that is normally in an essentially planar open configuration of dimensions sufficiently large to support said surgical patch, which upon exertion of pressure in said plane is compressed to a linear closed configuration, said linear closed configuration being of dimensions sufficiently small to enable insertion into a tubular member designed for use in minimally-invasive surgery, and which upon release of said pressure at least partially re-opens toward said open configuration. In some embodiments of the device, said linear closed configuration is of dimensions sufficiently small to enable insertion into a tubular member characterized by an internal diameter of between 5 mm and 12 mm. In some embodiments of the device, said linear closed configuration is of dimensions sufficiently small to enable insertion into a tubular member characterized by an internal diameter of 5 mm. In some embodiments of the invention, the tubular member is selected from the group consisting of cannulas, trocars, and tubes.

It is a further object of this invention to disclose embodiments of the device in which said base comprises a frame, wherein said frame comprises: two flexible points or regions (130) opposing one another which, upon said exertion of pressure essentially perpendicular to a line connecting said two flexible points or regions, collapse outward to form acute angles; and, corners (110) of sufficient flexibility such that upon said exertion of pressure expand outward to an essentially linear configuration.

It is a further object of this invention to disclose embodiments of the device in which said base comprises a frame, wherein at least one of the following is true: two opposing points or regions of said frame are thinned relative to the remainder of the frame, thereby providing flexibility to said frame; and, two opposing points or regions of said frame are angled outward such that under pressure applied in a direction generally perpendicular to a line connecting said two opposing points or regions, said two opposing points or regions would tend to buckle outward.

It is a further object of this invention to disclose the device as defined in any of the above, wherein said frame is adapted for attachment of said patch to said frame. In some embodiments of the invention in which said frame is adapted for attachment of said patch to said frame, said frame comprises a plurality of pins (140) extending out of the plane of said frame. In some embodiments of the invention in which said frame is adapted for attachment of said patch to said frame, said frame comprises a plurality of holders (360) pivotably attached to said frame, each of said holders comprising a leg and a head that extends essentially horizontally from said leg.

It is a further object of this invention to disclose embodiments of the device in which said base comprises a frame as defined in any of the above, wherein said frame comprises at least one depression along its outer perimeter.

It is a further object of this invention to disclose embodiments of the device in which said base comprises a frame as defined in any of the above, wherein said frame comprises at least one tab or handle (120) extending outward from said frame. In some preferred embodiments of the invention, said tab or handle is essentially in the plane of said frame.

It is a further object of this invention to disclose embodiments of the device as defined in any of the above, wherein said frame (100) comprises: a lower portion (500); an upper portion (510) of shape and dimensions substantially identical to those of said lower portion; a hinge element (520) providing a hinge connection between said lower portion and said upper portion such that rotation about said hinge connection brings said frame into a closed-jaw configuration in which said lower portion lies atop said upper portion and said upper portion and said lower portion and are substantially congruent with one another; a mechanism for reversibly locking said lower portion and said upper portion together; tongues (560) on two opposing sides of said upper portion; and, grooves (550) on two opposing sides of said lower portion, said grooves of dimensions to allow each tongue to be inserted into a matching groove, and positioned such that when said frame is in said closed-jaw configuration, each of said tongues engages a matching groove.

In some preferred embodiments of the invention in which the frame comprises a lower portion and an upper portion, said mechanism for reversibly locking said lower portion and said upper portion together comprises a plurality of pins (530) located on said lower portion and a plurality of holes (540) located on said upper portion, said holes having dimensions large enough to allow said pins to pass therethrough, and located such that said pins engage said holes when said frame is in said closed-jaw configuration. In some particularly preferred embodiments, each of said pins comprises a lip or protrusion; the distance from said lower portion to said lip or protrusion is at least equal to a thickness of said upper portion; and said holes are intersected by slots.

In some preferred embodiments of the invention in which the frame comprises a lower portion and an upper portion, said frame comprises corners (110) that incorporate an indentation.

It is a further object of this invention to disclose embodiments of the device in which said base comprises a frame as defined in any of the above, wherein said frame comprises a biocompatible plastic.

It is a further object of this invention to disclose embodiments of the device in which said base comprises a frame as defined in any of the above, wherein said frame comprises a biocompatible metal.

It is a further object of this invention to disclose embodiments of the device in which said base comprises a frame as defined in any of the above, wherein said frame additionally comprises at least one elongate member (490), said elongate member attached to two opposing sides of said frame, and curved out of said plane of said frame.

It is therefore one object of this invention to disclose a device for introducing a self-adhesive surgical patch during minimally invasive surgery, said device comprising a flexible base (700) constructed from a biocompatible elastomer and a roller (710) attached to said flexible base, wherein said base is sufficiently flexible to be compressible to a size sufficiently small to be capable of insertion into a tubular member designed for use in minimally-invasive surgery. In preferred embodiments of the invention, the base is sufficiently thin that when rolled into a substantially cylindrical shape, said cylindrical shape has a cross section characterized by a maximum transverse dimension not exceeding 5 mm.

It is a further object of this invention to disclose the device as defined in any of the above, wherein said device additionally comprises a pouch (600) that can adopt rolled and unrolled configurations and configured to be unrolled to enclose said frame when said frame is in said closed configuration. In some preferred embodiments of the invention, said pouch is attached to said frame. In some preferred embodiments of the invention in which said device comprises a pouch, said device additionally encloses a flexible band (610) adapted to roll or tear said pouch when said pouch is in said unrolled configuration. In some preferred embodiments of the invention, said flexible band comprises a thread. In some preferred embodiments of the invention, said flexible band comprises a ribbon made from a flexible biocompatible elastomer.

It is a further object of this invention to disclose the device as defined in any of the above, wherein said device is adapted for introduction of a self-adhesive hemostatic patch during minimally invasive surgery.

It is a further object of this invention to disclose the device as defined in any of the above, wherein said frame wherein said frame comprises sides having a cross-sectional shape and size selected from the group consisting of: a substantially rectangular cross section characterized by sides that not exceeding 2.5 mm in length; a substantially circular cross section characterized by a diameter not exceeding 2.5 mm; a substantially elliptical or oval cross section characterized by a major axis not exceeding 2.5 mm; a substantially polygonal n-sided cross section (n≠4); and, any combination of the above.

It is a further object of this invention to disclose a method for applying a self-adhesive surgical patch during minimally invasive surgery, wherein said method comprises:
if said device comprises a frame: (a) affixing said self-adhesive patch to said frame above while said frame is in said open configuration, thereby forming a patch/frame assembly; (b) applying pressure to at least two opposing sides of said patch/frame assembly, thereby transforming said patch/frame assembly to said closed configuration;
if said device comprises a thin flexible base: (a) placing said self-adhesive patch on a surface of said base, thereby forming a patch/base assembly; (b) rolling said patch/base assembly into a cylinder sufficiently small to be capable of introduction into a tubular member designed for use in minimally-invasive surgery;
(c) introducing said patch/frame assembly or said patch/base assembly via a tubular member; (d1) if said device comprises a frame, re-opening said patch/frame assembly to said open configuration; (d2) if said device comprises a thin flexible base, unrolling said patch/base assembly; (e) placing said patch on a target tissue or organ; and, (f) detaching said frame or base from said patch.

It is a further object of this invention to disclose such a method, wherein said step of placing said patch on a target tissue or organ is followed by a step of holding said patch/frame assembly in place until said self-adhesive patch at least partially adheres to said target tissue or organ.

It is a further object to disclose the method as defined in any of the above, wherein said device comprises an extendable pouch, and said step (b) of applying pressure to at least two opposing sides of said patch/frame assembly, thereby transforming said patch/frame assembly to said closed configuration, or rolling said patch/base assembly into a cylinder sufficiently small to be capable of introduction into a tubular member designed for use in minimally-invasive surgery is followed by a step of unrolling said extendable pouch so as to enclose said patch/frame assembly or said patch/base assembly; and said step introducing said patch/frame or said patch/base assembly is followed by a step of re-rolling or tearing said pouch, thereby exposing said patch/frame assembly or said patch/base assembly.

It is a further object of this invention to disclose the method as defined in any of the above, wherein said self-adhesive surgical patch is a self-adhesive hemostatic patch.

If is a further object of this invention to disclose the method as defined in any of the above, wherein said step of placing said patch on a target tissue or organ is followed by a step of holding said patch/frame assembly in place until said self-adhesive patch at least partially adheres to said target tissue or organ.

It is a further object of this invention to disclose the device as defined in any of the above, for use in the method as defined in any of the above. It is a further object of this invention to disclose the use of the device as defined in any of the above in the method as defined in any of the above.

It is a further object of this invention to disclose a kit for supplying a device for introducing a self-adhesive surgical patch during minimally invasive surgery, said kit comprising: the device as defined in any of the above; a protective sleeve (800) of dimensions sufficient to enclose said frame when said frame is in a closed configuration; a thread (810) attached to said protective sleeve; a suture holder (820) configured to hold said thread prior to use of said device; an assisting tube (830) configured to assist rolling of said protective sleeve; and, a tray (840) configured to hold said device, said protective sleeve, said thread, said suture holder, and said assisting tube. In some preferred embodiments of the kit, it additionally comprises medical grade breathable paper that extends over said tray, thereby sealing said kit.

In some especially preferred embodiments of the kit, it comprises the device that comprises a frame that includes a lower portion (500) and an upper portion (510) as defined in any of the above.

### BRIEF DESCRIPTION OF THE DRAWINGS

The invention will now be described with reference to the drawings, wherein:

FIG. 1 comprises schematic drawings showing several views of one non-limiting embodiment of a frame for delivering a surgical patch according to the invention disclosed herein;

FIGS. 2A and 2B illustrate schematically the transformation of one embodiment of the frame disclosed herein between an open (planar) and a closed (linear) configuration;

FIG. 3 comprises schematic drawings showing several views of another non-limiting embodiment of a frame for delivering a surgical patch according to the invention disclosed herein;

FIGS. 4A and 4B illustrate schematically a non-limiting embodiment of a frame for delivering a patch in which the frame includes pivotable holders for securing the patch to the frame, with the holders positioned to allow placement of the patch on the frame and to grasp the patch, respectively;

FIGS. 5A and 5B illustrate schematically top and side views, respectively, of an embodiment of the frame for delivering a surgical patch of according to the invention disclosed herein in which the frame incorporates an element designed to place pressure on the surgical patch when the patch is applied to tissue;

FIGS. 6A-6F illustrate schematically several views of another embodiment of the frame for delivering a surgical patch according to the invention disclosed herein in which the frame is of a jaw-like construction;

FIG. 7 illustrates schematically a non-limiting embodiment of the invention in which it includes an extendable pouch that can be unrolled to enclose the frame when it is in its linear configuration, and a flexible band for removing or tearing the pouch after the frame assembly has exited the tubular member through which it is introduced;

FIGS. 8A-8H illustrate the use of the pouch shown schematically in FIG. 7 to enclose and introduce a patch/frame assembly of the invention disclosed herein;

FIGS. 9A-9D illustrate schematically several views of a non-limiting embodiment of the instant invention in which the patch sits on a flexible base that is rolled to a cylinder prior to its introduction into the body of the patient; and,

FIGS. 10A and 10B illustrate schematically exploded and collapsed views, respectively, of a kit comprising components for delivering a surgical patch during minimally invasive surgery; and,

FIGS. 11A-11H illustrate schematically the use of the kit shown in FIG. 10 to pass a hemostatic patch through a 5 mm i.d. trocar.

It is emphasized that the figures are presented for illustrative purposes only, in order to assist a person of ordinary skill in the art to make and use the invention disclosed herein. Unless explicitly described to the contrary in the specification, none of the specific shapes, specific dimensions, or specific relative shapes or dimensions illustrated schematically in the figures are to be taken as being in any way limiting.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore, the invention is not limited by that which is illustrated in the figures and described in the specification, but only as indicated in the accompanying claims, with the proper scope determined only by the broadest reasonable interpretation of said claims. In some cases, for clarity or conciseness, individual elements of the invention are discussed separately. Nonetheless, any combination of individual elements of the invention disclosed herein that is not self-contradictory is considered by the inventors to be within the scope of the invention.

In all cases in which an embodiment is described as “comprising” a set of components or method steps, i.e. the invention may include components or method steps in addition to those explicitly listed, the scope of invention is to be understood to include embodiments in which the invention “consists of” the listed components or method steps, i.e. embodiments that include the listed components or method steps and no others, and to include as well embodiments in which the invention “consists essentially of” the listed components or method steps, i.e. embodiments that do not include any components or method steps not listed that would materially affect the basic and novel characteristics of the invention.

Unless specifically stated otherwise, any numerical range disclosed herein is understood to include within its scope any subrange. As non-limiting examples, if a range is stated to be “1-10%,” ranges of 1-5%, 2-9%, etc., are considered by the inventors to be within the scope of the invention; similarly, if a range is stated to be “less than 50%,” ranges of less than 40%, less than 25%, less than 10%, etc., are considered to be within the scope of the invention.

As used herein, with reference to numerical quantities, the expression “about” refers to a tolerance of ±25% about the nominal value.

In the descriptions and illustrations of the various embodiments of the invention disclosed herein, like reference numbers refer to like parts of the invention.

As used herein, with reference to surgical patches, the term “self-adhesive” refers to a patch that adheres to tissue or to an organ without any need for addition of any substance that will attach or adhere the patch to the tissue or organ and without any need for means for attachment that are external to the patch such as suturing or stapling. Unless otherwise specified, patches to which an adhesive is affixed or embedded as part of the manufacturing process as well as patches to which an adhesive is applied after manufacture at any time prior to attachment to a target tissue or organ are within the scope of this definition.

As used herein, the term “tubular member” refers to an elongated structure with at least one passage passing entirely therethrough along the structure's long axis. Non-limiting examples of tubular members include trocars, cannulas, and tubes.

As used herein, the term “polygonal” is used to describe a shape that has at least three essentially straight sides that together form a closed essentially planar structure. Non-limiting examples of structures that are to be considered “polygonal” within the scope of the term as it is used herein include closed figures with straight sides and vertices; structures with rounded corners; structures with rounded sides; structures with curved sides; and structures that have sides with indentations or extrusions.

For simplicity and clarity of presentation, the term “device” is used generically to describe the invention disclosed herein. The use of this term is not to be construed as an assertion, admission, or concession regarding whether any embodiment of the invention disclosed herein would be considered to be a “device” or to be an “accessory” as these terms are defined by the United States Food and Drug Administration, and no such assertion, admission, or concession, should be inferred from the use herein of the term “device” in any description of the invention. Nor should the use of the term “device” be understood as necessarily excluding “accessory” from the scope of any claim.

The instant invention discloses novel devices and methods for introducing a self-adhesive surgical patch such as a hemostatic patch during minimally-invasive surgery in which the patch is reversibly attached to a base that can be reduced in size to fit within a tubular member designed for use in minimally-invasive surgery. In typical non-limiting embodiments of the invention, the base can be reduced in size to fit within a tubular member having an internal diameter of 5-12 mm. In preferred embodiments of the invention, the base can be reduced in size to fit within a 5 mm i.d. tubular member such as a 5 mm i.d. trocar. After the patch/base assembly is introduced through the tubular member (e.g., the trocar), the assembly is restored to its original size, the patch applied to the target tissue or organ, and the base removed. In some embodiments of the invention, the base is a frame made of a compliant material to which the patch is attached at least two of its edges, while in other embodiments, the base is a thin flexible support of dimensions at least as large as the patch upon which the patch sits prior to its deployment. In some embodiments of the invention, the invention is adapted for introduction of a hemostatic patch. In some preferred embodiments of the invention, it is adapted for introduction of a hemostatic patch that uses a thrombin/fibrinogen sealant to attach the patch to a target organ or tissue. In some particularly preferred embodiments of the invention, the invention is adapted for introduction of a hemostatic patch of the type disclosed in U.S. Pat. Nos. 10,905,792, 11,071,805, and 11,471,556, and 11,771,799.

In some preferred embodiments of the invention disclosed herein, the device for delivering the surgical patch comprises a frame (100) that is made of a compliant material. That is, the material from which the frame is constructed allows it to be compressed when a force is applied to it and to return at least partially to its original configuration when the force is released. The surgical patch is attached to and delivered from the frame, as described in detail below. The frame may be made of any biocompatible compliant material that is sufficiently stiff to support the patch, but sufficiently flexible to be able to open and close as described below. Non-limiting examples of suitable materials include biocompatible plastics such as medical grades of PVC, polyethylene, PET, polypropylene, polycarbonate, PEEK, polysulfone, and polyurethane, and biocompatible metals such as stainless steel and nitinol.

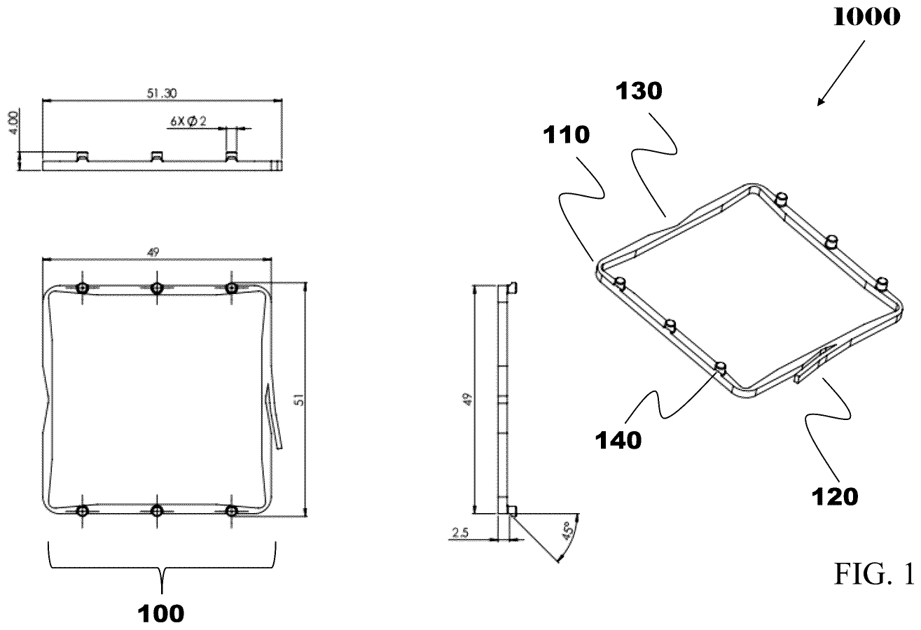

Reference is now made to FIG. 1, which presents schematic drawings showing several views of one non-limiting embodiment 1000 of the device for delivering a surgical patch disclosed herein. In the embodiment shown, the frame (100) is essentially square with sides of length approximately 50 mm. In typical embodiments, the sides of the frame have a cross-sectional shape that is substantially rectangular, preferably square. Other shapes, non-limiting examples of which include cross-sectional shapes having rounded corners, cross-sectional shapes having rounded or curved sides, generally rounded shapes such as circles and ellipses, and n-sided substantially polygonal shapes (n≠4), are all considered by the inventors to be within the scope of the invention. In embodiments in which the sides of the frame are substantially rectangular, the width and depth of the frame (i.e., the lengths of the sides of the rectangular cross sections) are typically 2-2.5 mm. The corners of the frame (110) are sufficiently flexible so that when pressure is applied to two opposing sides of the frame, two diagonally opposed corners open sufficiently, and the two remaining corners close sufficiently, that the frame transforms from the polygonal, essentially planar, “open” configuration shown in the figure into a “closed” configuration in which the frame adopts an essentially linear structure.

The frame can thus be inserted, while in its closed configuration, into a tubular member such as a trocar designed for use in minimally-invasive surgery. In those preferred embodiments in which the sides the frame are characterized by a dimension width and depth (e.g., the lengths of the sides of the frame or its maximum cross-sectional diameter) of no more than 2.5 mm, the frame in its closed configuration will thus have a transverse dimension not exceeding 5 mm, and can therefore be inserted in its closed configuration into a 5 mm i.d. tubular member such as a trocar. While in these preferred embodiments, the frame can be inserted in its closed configuration into a trocar characterized by an interior diameter of 5 mm, frames adapted to be inserted into tubular members of other interior diameters (non-limiting examples of which include standard trocars characterized by an i.d. of 5-12 mm such as 6 mm i.d. trocars, 10 mm i.d. trocars, and 12 mm i.d. trocars) are considered by the inventors to be within the scope of the invention. In the embodiment shown in FIG. 1, in order to make it easier for the surgeon to grasp and squeeze the frame using a standard grasper used in minimally invasive surgery, the frame incorporates at least one tab or handle 120 that extends outward from the frame. In preferred embodiments of the invention, the tab or handle is essentially in the plane of the frame, but configurations in which the tab is not coplanar with the frame are considered by the inventors to be within the scope of the invention.

The frame can be manufactured by any method known in the art for producing structures of the desired size and shape from the chosen material. Non-limiting examples include injection molding, 3-D printing, and extrusion.

The embodiment of the frame illustrated in FIG. 1 includes a plurality of pins 140 that extend from one side of the frame. These pins are for affixing the surgical patch to the frame. The patch is extended over the frame, and pulled over the pins so that they penetrate into or puncture the patch, thereby holding the patch to the sides of the frame while leaving the center of the patch undisturbed. In the embodiment illustrated in the figure, the frame incorporates a total of six pins, three on each of two opposing sides. In this embodiment, the pins are characterized by a height and depth of about 2-2.5 mm. As shown in the figure, in typical embodiments of the invention, the pins are not perfectly cylindrical, but rather include an extension or lip at the top in order better to grasp the patch when it is placed on the frame. It is emphasized that the number and exact size and shape of the pins should not be considered to be limited by the drawing; in embodiments in which the patch is affixed to the frame by use of pins, any convenient number of pins, of any size sufficiently large to enable affixing the patch to the frame and sufficiently small to allow the frame to fit into a tubular member designed for use in minimally-invasive surgery in the frame's closed configuration, and any convenient shape for attaching the patch, may be used.

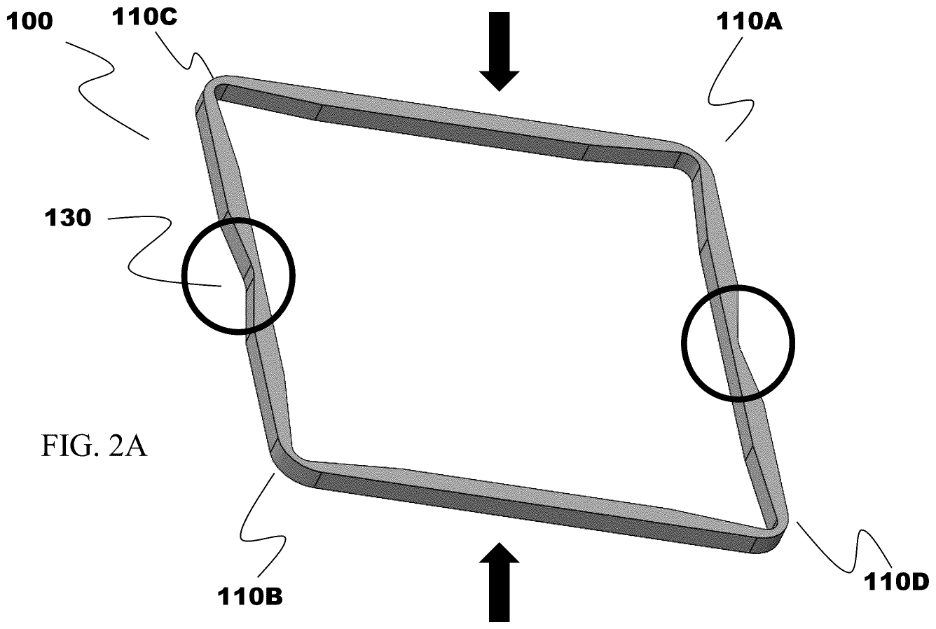

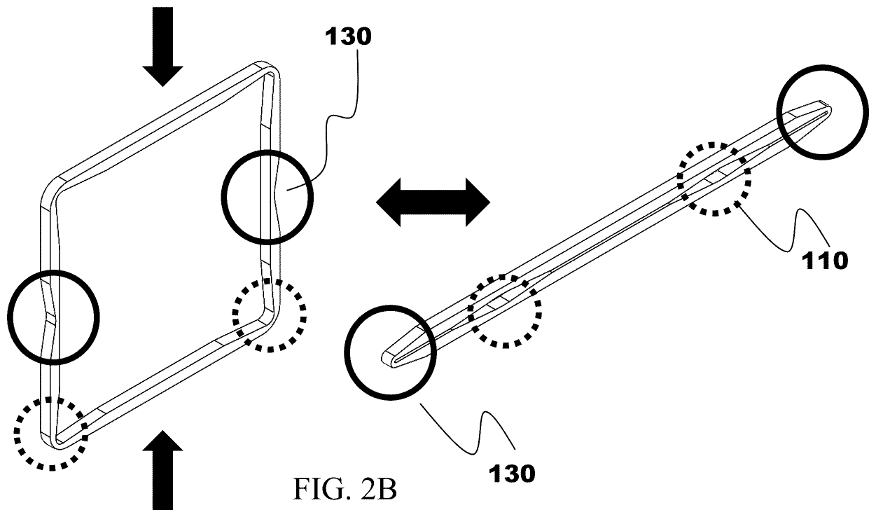

Reference is now made to FIGS. 2A and 2B, which illustrate schematically the transformation between the open and closed configurations of a typical non-limiting embodiment of the frame. In the non-limiting embodiment illustrated in the figure, the frame comprises two flexible points or regions 130 on opposing sides. The flexibility may be provided by providing an indentation in the frame at these two points, as is illustrated in FIG. 2A; the two flexible points are indicated by the circles. In some preferred embodiments of the invention, the frame is slightly angled outward at these points so that they will tend to collapse outward rather than inward. As illustrated schematically in FIG. 2A, the frame is transformed from its planar open configuration to its closed linear configuration by application of pressure to the two opposing sides of the frame that do not include the flexible points, as indicated by the arrows. As shown in FIG. 2B, when pressure is applied in this manner, the two flexible points or region collapse outward to form acute angles, as indicated by the solid circles, which approach an angle of zero degrees in the limit of complete collapse, while the corners of the frame expand to an essentially linear configuration, with the change for two adjacent angles indicated by the dashed circles. When the pressure is released, the frame, being made of a compliant material, at least partially returns to its essentially polygonal open structure. Embodiments in which upon release of the pressure, the frame only partially reopens, and is fully restored to its open configuration by grasping the partially open frame and pulling outward, thereby returning the frame to its fully open configuration, are considered by the inventors to be within the scope of the invention.

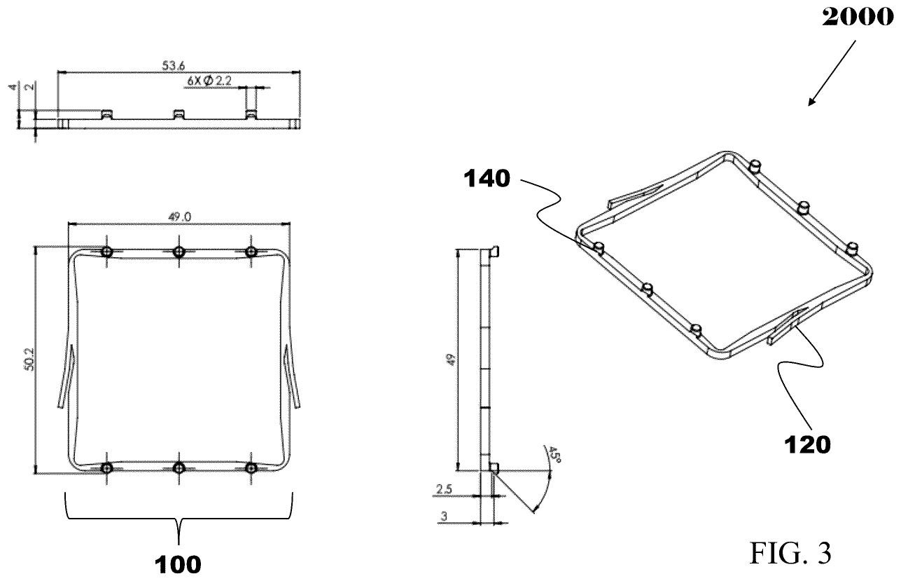

Reference is now made to FIG. 3, which presents schematic drawings showing several views of another non-limiting embodiment 2000 of the invention. The frame shown in FIG. 3 is essentially similar to those shown in FIGS. 1 and 2, except that rather than a concave depression in the outer perimeter of one side of the frame, two opposing sides of the frame have tabs 120 extending outward from the frame, which in preferred embodiments are essentially in the plane of the frame.

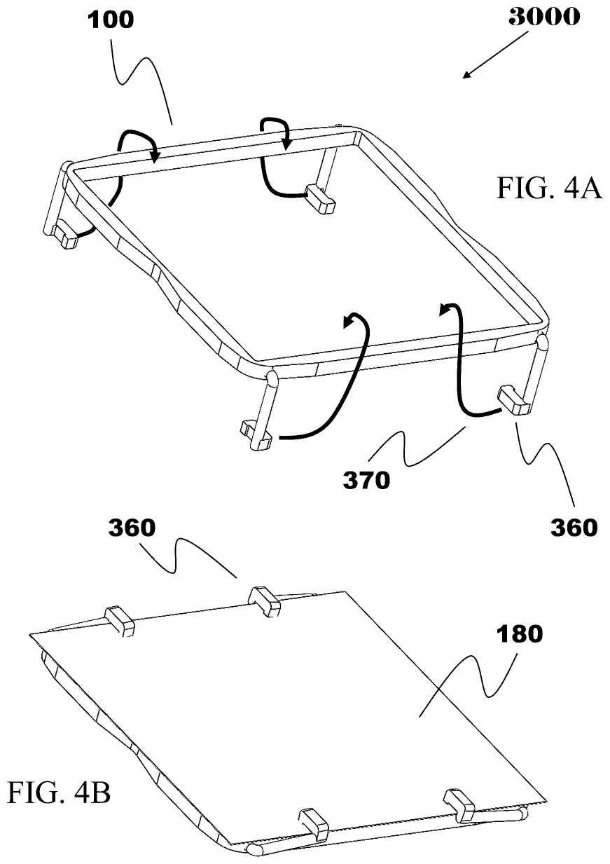

Reference is now made to FIG. 4, which illustrates a third non-limiting embodiment 3000 of the invention that incorporates an alternative mechanism for attaching a patch to the frame. In this embodiment, a plurality of pivotably attached holders 360 is attached to the external perimeter of the frame. A typical non-limiting embodiment is illustrated in FIG. 4A. In the embodiment illustrated, a plurality of holders (four in the embodiment illustrated) are pivotably attached to the external perimeter of the frame. The direction of the pivot of the holders is illustrated by arrows 370 in the figure. The holders comprise a leg and a head that extends essentially horizontally from the head. In preferred embodiments of the invention, the head has a concavity such that when the leg is rotated about the pivot point on the frame, the head can grasp or attach to the frame. A configuration in which the holders have been pivoted to hold a patch 180 onto the frame is illustrated in FIG. 4B. When the surgeon wishes to deploy the patch, the holders are swiveled back to a position in which they do not grasp the patch, and the patch is removed from the frame. This embodiment of the means for attaching the patch has the advantage that the patch can be attached to and removed from the frame without puncturing the patch.

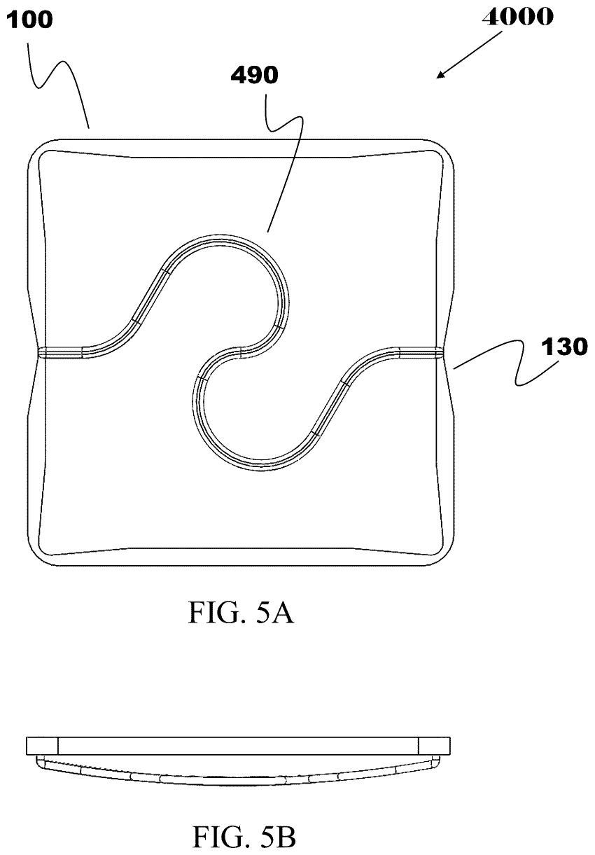

Many self-adhesive surgical patches, particularly those in which the adhesive is activated by contact with bodily fluids require application of pressure for a short time after their delivery in order to ensure that they will adhere fully to the tissue or organ to which they are delivered. Reference is now made to FIG. 5, which provides a schematic illustration of one non-limiting embodiment 4000 of the frame of the invention disclosed herein in which the frame incorporates an element that provides such pressure. A top view of a typical configuration is shown in FIG. 5A. The frame incorporates a flexible elongated element 490 that is attached to two opposing sides of the frame. In the embodiment illustrated in the figure, the element is curved along its length roughly into an “S” shape. This type of shape is preferred as it maximizes contact with the patch, but should not be considered limiting in any way; the element may adopt any convenient shape that allows the frame to be transformed to a linear closed configuration that will fit into a tubular member designed for use in minimally-invasive surgery such as a 5 mm i.d. trocar. A side view of the frame according to this embodiment is illustrated schematically in FIG. 5B. As can be seen from the figure, the elongated element does not lie in the plane of the frame, but rather extends outside of the plane in a bowed configuration. When the system is assembled, the patch is placed on the side of the frame on which the elongated element rises outside of the plane of the frame, preferably sufficiently tightly such that the elongated element at least partially deforms in the direction of the plane of the frame. When the patch is delivered, the elongated element will place pressure on the patch while the frame is held in place at the site of delivery of the patch. Once the adhesive of the patch has activated sufficiently for it to adhere to the tissue to which it is applied, the frame can be removed.

Insertion and application of the patch can be performed as follows. The patch is attached to the frame as described above, with the adhesive side of the patch facing upward, that is, the adhesive side of the patch is not in direct physical contact with the frame, thereby forming a patch/frame assembly. This step can be performed in a sterile environment such as a clean room and the patch/frame assembly placed in a sterile package for use in the surgery, or the assembly can be performed in the operating room prior to its use in minimally invasive surgery. The assembly is then collapsed to the frame's closed linear configuration, and the assembly introduced into the patient's body via a tubular member designed for use in minimally-invasive surgery (e.g., a 5 mm i.d. trocar). After the assembly has been inserted, the release of pressure on the sides of the frame will cause the frame to reopen at least partially toward its open configuration. In some embodiments of the invention, the frame is made of a material that does not fully return to the open configuration automatically. In these embodiments, the frame is reopened by the use of a pair of graspers. The patch/frame assembly is then maneuvered to the site of application and placed upon the tissue or organ to which the patch is to be applied with the patch side of the assembly contacting the tissue or organ. In preferred embodiments of the invention, the adhesive is activated at this point, e.g., by reaction with bodily fluids on the surface of or exuded from the tissue. Once the patch has adhered to the tissue sufficiently, the frame is detached, leaving the patch behind, recompressed to its linear configuration, and removed via a tubular member designed for use in minimally-invasive surgery (generally, but not necessarily, the tubular member through which it was introduced).

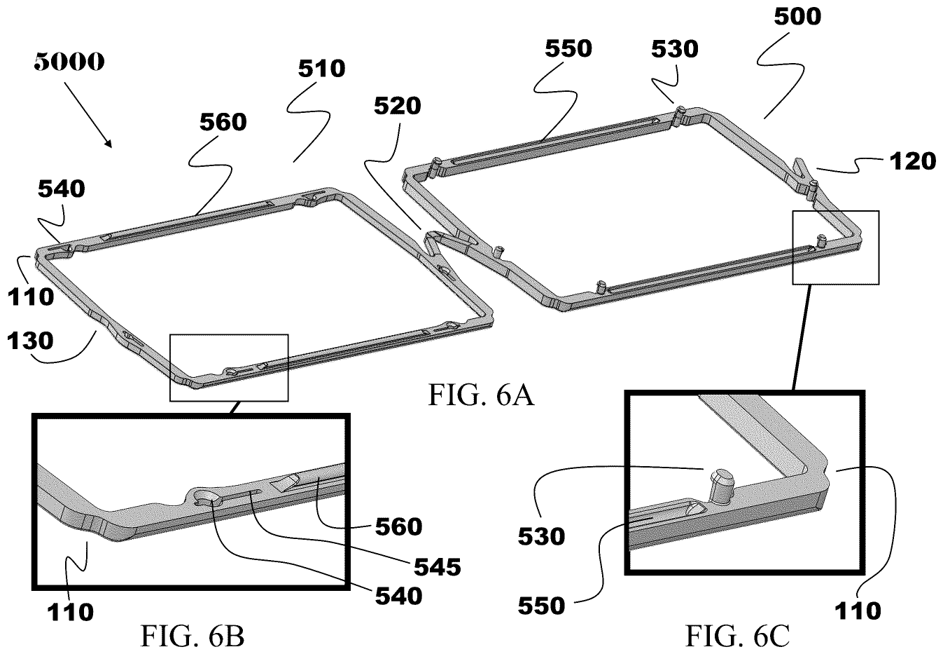

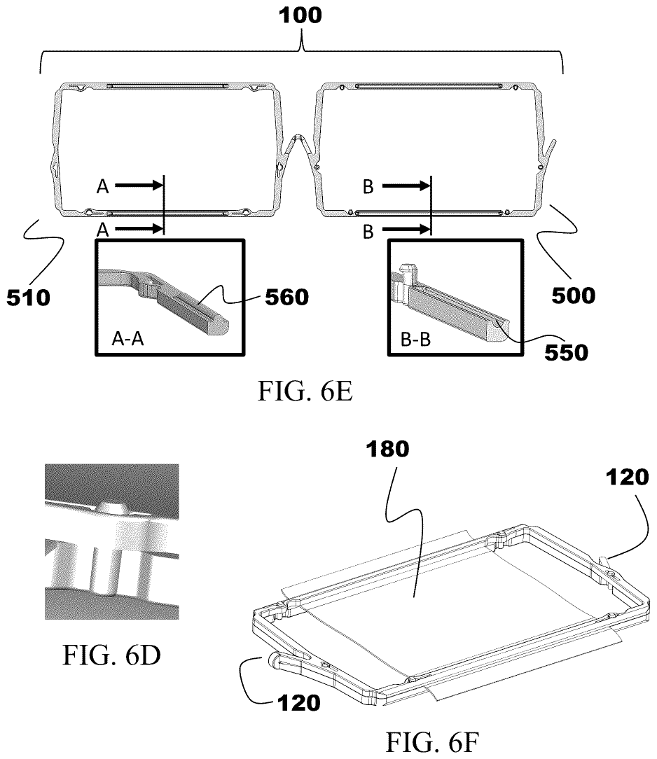

Reference is now made to FIG. 6, which presents several views of an additional embodiment 5000 of the frame of the invention disclosed herein in which the frame has a jaw-like configuration in which the frame comprises two parts that are in an open-jaw configuration prior to the placement of the patch and which, after placement of the patch on the frame, are then closed one upon the other in order to a closed-jaw configuration that holds the patch. Reference is now made to FIG. 6A, which illustrates schematically a preferred embodiment of the invention. The frame (100) comprises two portions of essentially the same shape and dimensions, a lower portion 500 and an upper portion 510. In a typical non-limiting embodiment of the invention, lower portion 500 and upper portion 510 are each essentially rectangular and of dimensions of about 5×7 cm when the frame is in its open configuration (i.e. prior to its compression into its closed configuration for insertion into the tubular member). Lower portion 500 and upper portion 510 are hingeably connected with a hinge element 520 that allows the upper and lower sections to rotate relative to one another about an axis that passes through the connector while substantially maintaining their positions relative to the plane perpendicular to the axis. Hinge element 520 need not be an actual hinge, but can be any flexible structure that allows relative motion of the two sections of the frame. As a non-limiting example, in the embodiment illustrated schematically in FIG. 6A, the hinge element comprises a piece of flexible plastic that connects the two sections of the frame. In preferred embodiments of the invention, lower portion 500 comprises a tab or handle 120, substantially coplanar with lower portion 500, on the side opposite to the side connected to the hinge element. In preferred embodiments of the invention, the hinge element incorporates a slot such that when the upper and lower portions of the frame are engaged one to the other, the hinge element forms a second tab or handle. Such an embodiment is illustrated schematically in FIG. 6A.

In preferred embodiments, the frame comprises a mechanism for reversibly locking the upper and lower portions together once they are brought together after rotating about the hinge element. A non-limiting example of a reversible locking mechanism is illustrated in FIG. 6A. The lower section of the frame includes a plurality of pins 530 that are disposed about the perimeter of the frame. It is emphasized that, in contrast to embodiments of the invention such as those illustrated in FIGS. 1 and 3, pins 530 do not engage the patch. On the sides of the lower portion of the frame that are adjacent to the side connected to the hinge element, the pins are separated by a distance longer than one dimension of the patch so that the patch can sit on the lower portion of the frame without being punctured by or otherwise engaged by pins 530. The upper portion of the frame comprises a plurality of holes 540 that are of a dimension and spacing that enables them to engage the pins such that when the pins engage the holes, the two portions of the frame sit one atop the other with their perimeters substantially congruent to one another. Enlarged views of the holes and pins are illustrated in FIGS. 6B and 6C, respectively. In preferred embodiments of the invention, as illustrated in FIG. 6C, the pins are not cylindrical, but include a lip or protrusion. In preferred embodiments of the invention, holes 540 pass all the way through the upper portion of the frame and are intersected by slots 545. Slot 545 gives the area around hole 540 sufficient flexibility that the pin can pass therethrough without damaging the upper portion of the frame. In embodiments such as the one depicted in FIG. 6, the reversible locking mechanism works by passing the pins through the matching holes until the lip at the top of the pin emerges and engages the upper portion of the frame. Reference is now made to FIG. 6D, which shows an expanded view of the pin and hole when the frame is in its closed and locked configuration.

In preferred embodiments of the jaw-like embodiment (5000) of the invention, corners 110 of the upper and lower portions of the frame incorporate an indentation to increase their flexibility and thereby enable the user to compress the frame more easily to a dimension that will fit into a tubular member designed for use in minimally-invasive surgery, preferably into a 5 mm i.d. trocar. Expanded views of the corners according to this embodiment are illustrated schematically in FIGS. 6B and 6C.

In order to hold the patch in place, the lower portion of the frame comprises grooves 550 on opposing sides of the frame, preferably the sides adjacent to the side attached to the hinge element. In preferred embodiments of the invention, the length of the grooves is at least equal to one dimension of the patch to be applied. The upper portion of the frame comprises tongues 560 on opposing sides of the frame that are not longer than the grooves, and are positioned such that when the two portions of the frame are brought into contact by rotation about the hinge element, the tongues will engage the grooves.

Reference is now made to FIG. 6E, which provides a detailed cross-sectional schematic illustration of the tongue-and-groove system for holding the patch in the frame. The left side of the figure shows a view along cross-section A-A of the upper portion of the frame, and the right side of the figure shows a view along cross-section B-B of the lower portion of the frame. As can be seen in the figure, in preferred embodiments, the depth and width and of the grooves are at least equal to the height and width of the tongues. In the embodiment shown in FIG. 6D, the tongues and grooves have a generally cylindrical or hemi-cylindrical shape, but this specific shape should not be considered limiting; any shape for the grooves and tongues that enables them to engage one another, hold a patch in place, and be separated one from the other upon application of sufficient force, is considered by the inventors to be within the scope of the invention.

In order to hold the patch in place, the patch is placed across the lower portion of the frame such that it sits on the two grooves. The upper portion is then rotated about the axis defined by the hinge connector and thereby engages the lower portion, bringing the frame into its closed-jaw configuration. The two portions of the frame are locked together by the locking mechanism (e.g. the pins and matching holes described above), while the patch is held in place by the engagement of the tongues and grooves. Reference is now made to FIG. 6F, which illustrates the frame in its closed configuration, holding a patch between the two portions of the frame. In the embodiment illustrated in the figure, the hinge element is designed such that when the frame is in its closed configuration, the hinge element forms a second tab 120.

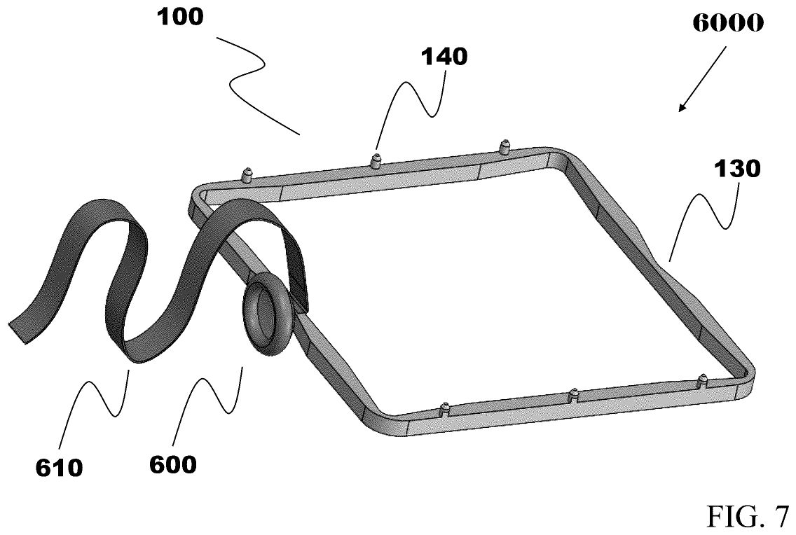

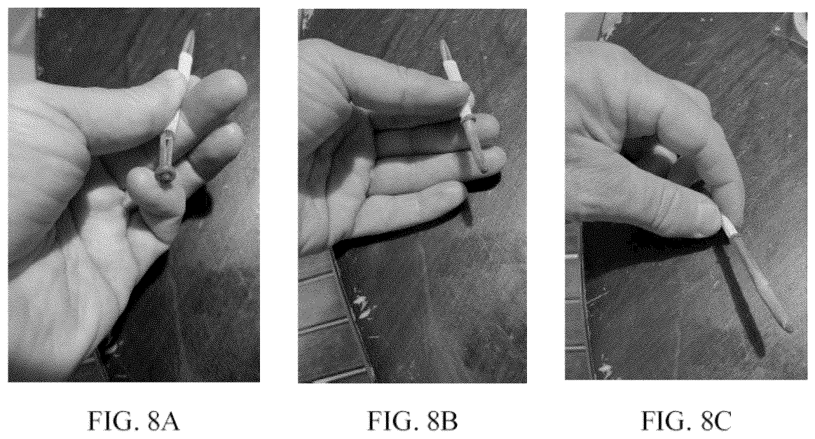

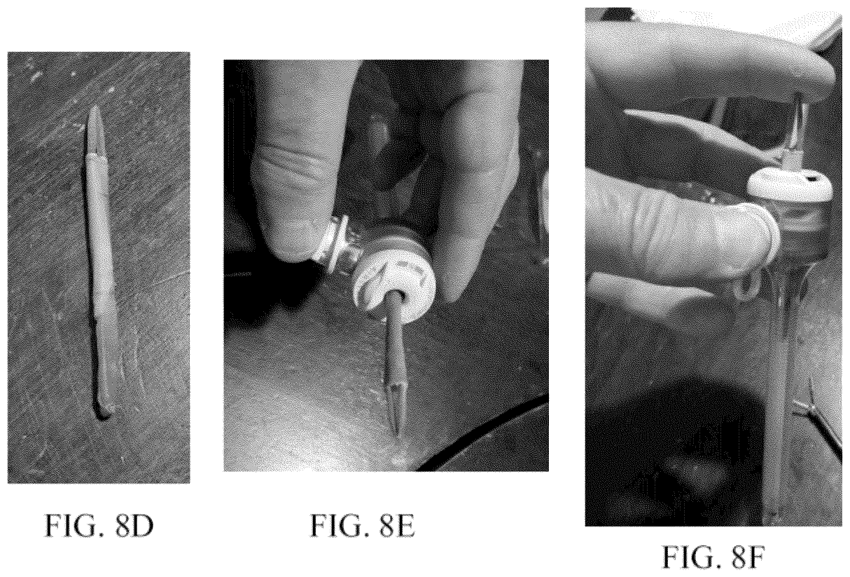

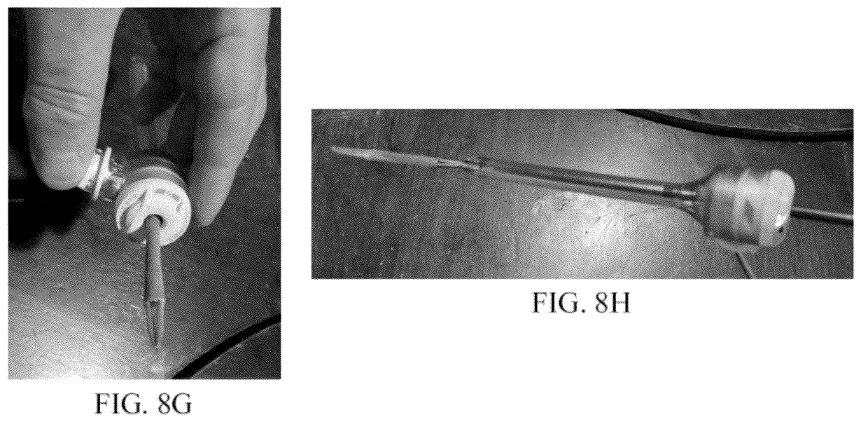

In order to simplify introduction of the patch/frame assembly into the patient's body during the surgery, some preferred embodiments of the invention include an extendable pouch into which the patch/frame assembly can be inserted prior to its introduction. Reference is now made to FIG. 7, which illustrates a non-limiting embodiment 6000 of the invention in which it includes such a pouch (600). In the embodiment illustrated in the figure, the pouch is physically attached to the frame, but embodiments in which it is an independent component of the system are considered by the inventors to be within the scope of the invention. The pouch can be made of any biocompatible elastomer that can be rolled into an essentially flat configuration and then unrolled to enclose the frame. Non-limiting examples of such elastomers include silicone, polyurethane, polyisoprene, and latex. The patch/frame assembly is compressed to its closed linear configuration, the pouch unrolled to enclose the assembly, and the pouch then inserted through the tubular member. Reference is now made to FIGS. 8A-8H, which illustrate sequential steps of a procedure for enclosure and introduction of the patch/frame assembly by using the pouch that is shown schematically in FIG. 7.

In some preferred embodiments of the invention in which the system includes an extendable pouch, a flexible band 610 that is designed to tear or unroll the pouch is provided as well. Such a band is illustrated schematically in FIG. 7. As with the pouch, the band may be attached to the frame, or included as a separate component. Non-limiting examples of designs of the flexible band include a thread and a ribbon made of a biocompatible substance such as polyethylene. After the assembly has been introduced into the patient's body, the flexible band is used to tear (in the case in which the band is in the form of a thread) or re-roll (in the case in which the band is in the form of a ribbon) the pouch, which can then be removed along with the frame once the patch has been deployed. In some preferred embodiments of the invention in which the flexible band comprises a thread, the pouch includes a weakened portion along its length to make tearing the pouch easier. As a non-limiting example, the pouch may be scored along its length.

It is also within the scope of the invention to disclose embodiments of the invention in which the base is a thin flexible solid base upon which the patch sits, rather than an open frame to which the patch is attached at its edges as described above. The base may be made of any biocompatible material that is sufficiently stiff to act as a stable base for the patch, but that is sufficiently flexible that when the patch is sitting on it, it can be rolled into a cylinder of outer diameter sufficiently small to fit inside a tubular member designed for use in minimally-invasive surgery. Non-limiting examples of materials from which the base can be made include biocompatible plastics and elastomers.

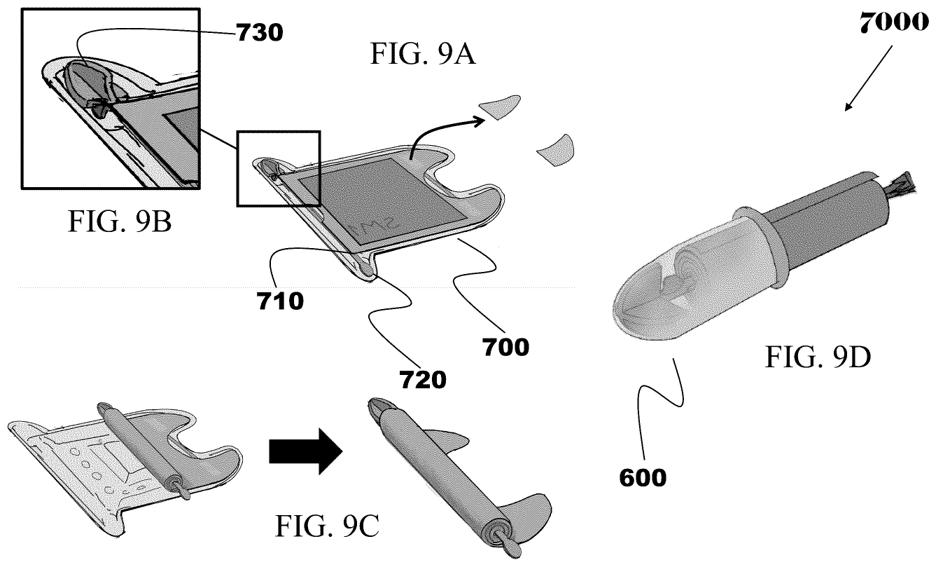

Reference is now made to FIGS. 9A-9D, which illustrate one non-limiting embodiment 7000 of the invention in which the frame comprises a thin solid base 700. As illustrated in FIG. 9A, a roller 710 is placed on the base at one side. In preferred embodiments of the invention, the base includes a depression 720 in which the roller sits. In preferred embodiments of the invention, the roller includes a head 730 of diameter less than 5 mm; an expanded view of the head is shown in FIG. 9B. The roller may be attached to the patch, e.g. via tabs or clips. The patch is placed on the base with the adhesive side upward, and then the patch rolled about the roller to form a cylinder of external diameter sufficiently small to fit into a tubular member designed for use in minimally-invasive surgery (e.g. a 5 mm i.d. trocar), as shown in FIG. 9C. In some preferred embodiments of the invention, the base includes a pouch of the type illustrated in FIGS. 7 and 8, which is extended over the rolled-up patch, thereby enclosing it for introduction into the body of the patient, as illustrated in FIG. 9D. The assembly is introduced via a tubular member such as a trocar, as described above. After the introduction of the assembly, the pouch (if used) is removed, the assembly unrolled using the roller, and the patch deployed on the target tissue or organ, where, in preferred embodiments of the invention, the adhesive is activated as disclosed above.

The inventors emphasize that while the figures illustrate embodiments of the invention in which the frame and patch are essentially rectangular, the figures are presented only to enable a person of ordinary skill in the art to make and use the invention disclosed herein, and are not to be considered to limit the size or dimensions of the base or patch in any way. The patch may be of any convenient shape, and the frame can be of any shape that is appropriate for delivering the patch and of any appropriate size that can be compressed to fit inside a tubular member designed for use in minimally-invasive surgery, preferably a tubular member characterized by a 5 mm i.d.

It is also within the scope of the invention to disclose a kit for providing a device for introducing a self-adhesive surgical patch during minimally invasive surgery according to the instant invention, along with components that can be used to assemble the device prior to its use.

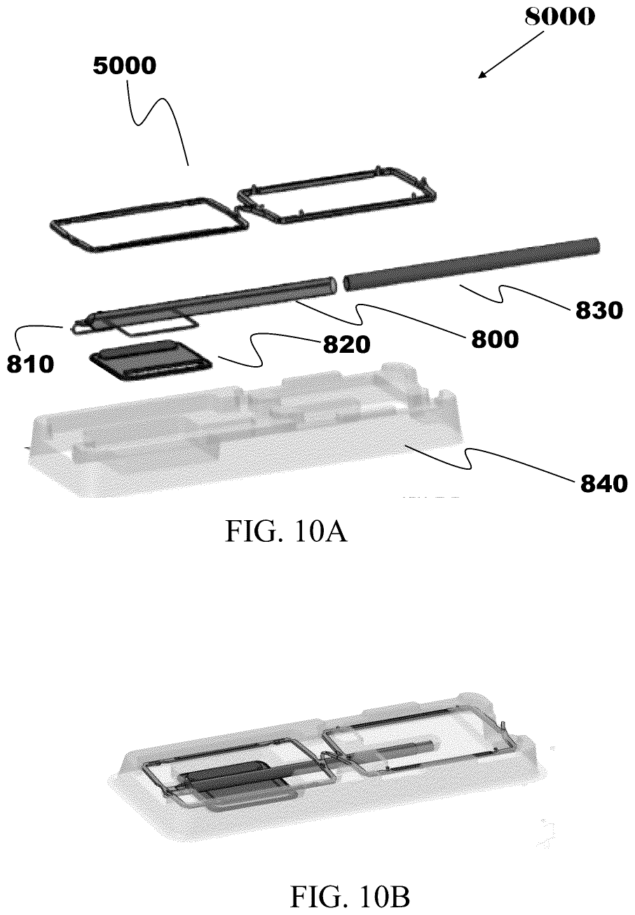

Reference is now made to FIG. 10A, which presents a schematic exploded view of one non-limiting embodiment (8000) of the kit disclosed herein. The kit comprises a frame (100); in the embodiment of the kit illustrated in FIG. 10, it comprises the embodiment of frame illustrated in FIG. 6 (5000). The kit further comprises a protective sleeve 800 that serves to protect the hemostatic patch from detrimental environmental effects such as exposure to fluids. In some non-limiting preferred embodiments of the invention, the protective sleeve is made of polyurethane. A thread or fiber 810 is attached to one end of the protective sleeve; the thread or fiber is used to extract the protective sleeve from the body of the patient once the patch has been introduced. In some non-limiting preferred embodiments of the invention, the thread or fiber is made of polyester. The kit also includes a suture holder 820 that holds the thread within the packaging. The suture holder does not contact the body of the patient; in some non-limiting preferred embodiments of the invention, the suture holder is made of polyethylene terephthalate glycol (PETG). The kit additionally comprises an assisting tube 830 that is used to assist the rolling of protective sleeve 800. In some non-limiting preferred embodiments of the invention, the assisting tube is made of polyether block amide, commercially available under trade names such as PEBAX and VESTAMID E. In the exploded view illustrated in FIG. 10A, the assisting tube is shown separated from the protective sleeve, but in preferred embodiments of the invention, the kit is provided with the assisting tube inserted into the protective sleeve. Finally, the kit comprises a tray 840 that includes indentations that are of a size and shape to permit the other components of the kit to rest securely therein. These indentations can be produced by any technique known in the art, e.g., by molding. The tray does not contact the body of the patient. In preferred embodiments of the invention, the tray is made from PETG, but it can be made from any appropriate material. In some embodiments, the kit includes a lid that slip-fits over the tray (the lid is not illustrated in FIG. 10A). In preferred non-limiting embodiments, the lid is made of PETG.

Reference is now made to FIG. 10B, which illustrates schematically one embodiment of the kit inside tray 840 as it is provided to the user. Once the components of the kit have been placed in the tray, the tray is sealed with a medical grade breathable paper, preferably a paper made of nonwoven HDPE fibers such as the paper marketed under the trade name TYVEK 1073B. The paper is designed to be peeled from the tray immediately prior to use of the elements of the kit. In preferred embodiments of the invention, the kit is sterilized prior to being supplied to the user, preferably by gamma irradiation to a sterility assurance level of 10⁻⁶ in accordance with ISO 11137-2:2013, optionally in conjunction with ISO 13004:2022.

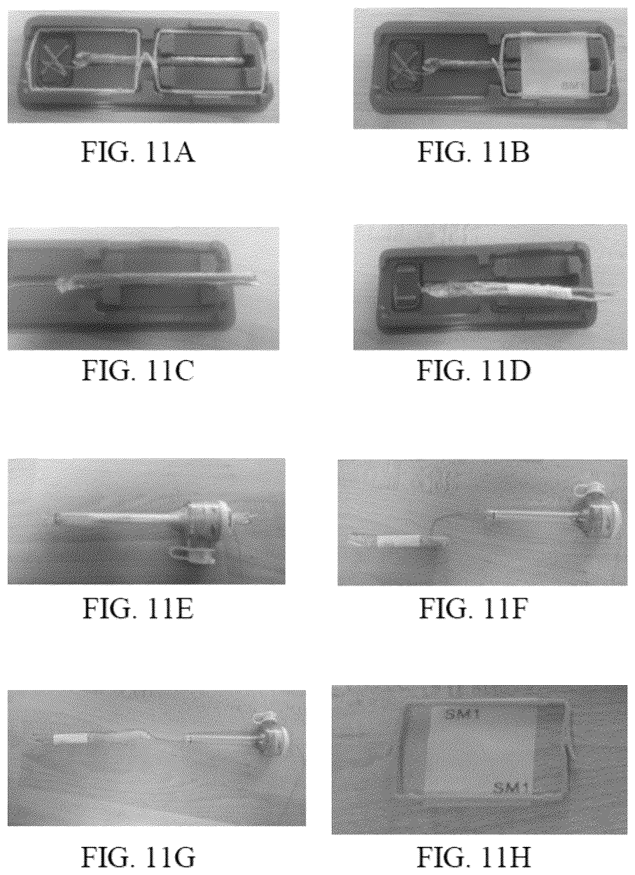

Reference is now made to FIG. 11, which illustrates a use of the kit illustrated in FIG. 10 to introduce a hemostatic patch into a 5 mm i.d. trocar. The illustrations in FIG. 11 are intended to assist a person of ordinary skill in the art to make and use the invention disclosed herein, and are not intended to be limiting in any way.

FIG. 11A illustrates one embodiment of the kit as it appears after the removal of the seal, with the components of the kit inside the matching depressions in the tray. In the embodiment illustrated in FIG. 11, a frame of the embodiment (5000) illustrated in FIG. 6 is used. A hemostatic patch is placed on the lower portion of the frame (e.g., by using a dry tweezer), and the frame is then closed to its closed-jaw configuration and locked, holding the patch in place, as illustrated in FIG. 11B. The frame is then collapsed to its closed configuration by pulling on tabs 120, thereby rolling the patch with the side that will engage the patient's tissue on the inner side of the roll. The frame is then inserted into the protective sleeve 800 by using the assisting tube 830 (FIG. 11C). Once the frame with the rolled film is placed in the protective sleeve, the assisting tube is removed and discarded, as illustrated in FIG. 11D. As shown in FIG. 11E, the frame (still inside the protective sleeve) is then inserted into a tubular member; in the embodiment illustrated, the protective sleeve and frame are inserted into a standard 5 mm i.d. trocar. The frame, still inside the protective sleeve, is then pushed through the trocar using any appropriate device known in the art such as an endoscope or grasper (FIG. 11F). Once the frame has been brought into position, the protective sleeve is removed by pulling on the thread or fiber 810, as illustrated in FIG. 11G. Once the protective sleeve has been removed, the frame opens toward its open configuration, with the patch held in place and ready for application on the desired tissue, as illustrated in FIG. 11H. Once the patch has been placed on the tissue and held in place for sufficient time for hemostasis to have occurred, the frame is detached from the patch using any appropriate tool such as a laparoscopic grasper, folded back into its closed configuration, removed from the body through the tubular member, and discarded.

1.-42. (canceled)

43. A device for introducing a self-adhesive surgical patch during minimally invasive surgery, said device comprising a flexible base, said base comprising a frame (100) which, when no pressure is applied, is in an essentially planar open configuration of dimensions sufficiently large to support said surgical patch, and which upon exertion of pressure in said plane is compressed to a linear closed configuration, said linear closed configuration being of dimensions sufficiently small to enable insertion into a tubular member designed for use in minimally-invasive surgery, and which upon release of said pressure at least partially re-opens toward said open configuration, wherein said frame comprises:
a lower portion (500);
an upper portion (510) of shape and dimensions substantially identical to those of said lower portion;
a hinge element (520) providing a hinge connection between said lower portion and said upper portion such that rotation about said hinge connection brings said frame into a closed-jaw configuration in which said lower portion lies atop said upper portion and said upper portion and said lower portion and are substantially congruent with one another;
a mechanism for reversibly locking said lower portion and said upper portion together;
tongues (560) on two opposing sides of said upper portion; and,
grooves (550) on two opposing sides of said lower portion, said grooves of dimensions to allow each tongue to be inserted into a matching groove, and positioned such that when said frame is in said closed-jaw configuration, each of said tongues engages a matching groove.

44. The device according to claim 43, wherein said mechanism for reversibly locking said lower portion and said upper portion together comprises:
a plurality of pins (530) located on said lower portion; and,
a plurality of holes (540) located on said upper portion, said holes having dimensions large enough to allow said pins to pass therethrough, and located such that said pins engage said holes when said frame is in said closed-jaw configuration.

45. The device according to claim 44, wherein:
each of said pins comprises a lip or protrusion;
the distance from said lower portion to said lip or protrusion is at least equal to a thickness of said upper portion; and,
said holes are intersected by slots.

46. The device according to claim 43, wherein said frame comprises corners (110) that incorporate an indentation.

47. The device according to claim 43, wherein said frame comprises:
two flexible points or regions (130) opposing one another which, upon said exertion of pressure essentially perpendicular to a line connecting said two flexible points or regions, collapse outward to form acute angles; and,
corners (110) of sufficient flexibility such that upon said exertion of pressure, said corners expand outward to an essentially linear configuration.

48. The device according to claim 43, wherein said frame comprises a material selected from the group consisting of biocompatible plastics and biocompatible metals.

49. The device according to claim 43, wherein said frame additionally comprises at least one elongate member (490), said elongate member attached to two opposing sides of said frame, and curved out of said plane of said frame.

50. The device according to claim 43, wherein said device additionally comprises an extendable pouch (600), said pouch configured to adopt rolled and unrolled configurations and configured to be unrolled to enclose said frame when said frame is in said closed configuration.

51. The device according to claim 50, wherein said pouch is attached to said frame.

52. The device according to claim 50, wherein said device additionally encloses a flexible band (610) adapted to roll or tear said pouch when said pouch is in said unrolled configuration.

53. The device according to claim 52, wherein said flexible band comprises at least one component selected from the group consisting of:
threads; and,
ribbons constructed from a flexible biocompatible elastomer.

54. The device according to claim 43, wherein said base comprises a solid base (700) and a roller (710) attached to said base, and said base is sufficiently flexible to be compressible to a size sufficiently small to be capable of insertion into a tubular member designed for use in minimally-invasive surgery.

55. A method for applying a self-adhesive surgical patch during minimally invasive surgery, said method comprising:
obtaining the device according to claim 43;
attaching said self-adhesive surgical patch to said device while said frame is in said open configuration, thereby forming a patch/frame assembly;
applying pressure to at least two opposing sides of said patch/frame assembly, thereby transforming said patch/frame assembly to said closed configuration;
introducing said patch/frame assembly into a patient's body via a tubular member;
re-opening said patch/frame assembly to said open configuration;
placing said patch on a target tissue or organ; and,
detaching said frame from said patch.

56. The method according to claim 55, wherein said step of placing said patch on a target tissue or organ is followed by a step of holding said patch/frame assembly in place until said self-adhesive patch at least partially adheres to said target tissue or organ.

57. The method according to claim 55, wherein:
said device comprises an extendable pouch (600) attached to said frame;
said step of applying pressure is followed by a step of unrolling said pouch so as to enclose said patch/frame assembly; and,
said step of introducing said patch/frame assembly is followed by a step of re-rolling or tearing said pouch, thereby exposing said patch/frame assembly and releasing said pressure.

58. The method according to claim 55, wherein:
said base of said device comprises a solid base (700) and a roller (710) attached to said base, and said base is sufficiently flexible to be compressible to a size sufficiently small to be capable of insertion into a tubular member designed for use in minimally-invasive surgery;
said step of rolling said patch/base assembly is followed by a step of unrolling said pouch so as to enclose said patch/base assembly; and,
said step of introducing said patch/base assembly is followed by a step of re-rolling or tearing said pouch, thereby exposing said patch/base assembly.

59. The method according to claim 55, wherein said step of attaching said self-adhesive surgical patch to said device while said frame is in said open configuration comprises:
placing said self-adhesive patch on said lower portion such that said self-adhesive patch lies atop said grooves;
rotating said upper portion about said hinge member such that said tongues engage said grooves, thereby holding said self-adhesive patch in place; and,
locking said frame into a closed-jaw configuration by use of said locking mechanism.

60. The method according to claim 55, wherein said self-adhesive surgical patch is a self-adhesive hemostatic patch.

61. A kit for supplying a device for introducing a self-adhesive surgical patch during minimally invasive surgery, said kit comprising:
the device according to claim 43;
a protective sleeve (800) of dimensions sufficient to enclose said frame when said frame is in a closed configuration;
a thread (810) attached to said protective sleeve;
a suture holder (820) configured to hold said thread prior to use of said device;
an assisting tube (830) configured to assist rolling of said protective sleeve; and,
a tray (840) configured to hold said device, said protective sleeve, said thread, said suture holder, and said assisting tube.

62. The kit according to claim 61, wherein said kit additionally comprises medical grade breathable paper that extends over said tray, thereby sealing said kit.
